(19) United States
(12) Patent Application Publication
(10) Pub. No.: US 2009/0197221 A1
(43) Pub. Date: Aug. 6, 2009

(54) DENTAL BONDING COMPOSITIONS AND METHODS USEFUL IN INHIBITION OF MICROLEAKAGE IN RESIN-BONDED DENTIN

(76) Inventors: Grayson W. Marshall, JR., Larkspur, CA (US); Sally J. Marshall, Larkspur, CA (US); Diana N. Zeiger, Germantown, MD (US); Antoni P. Tomsia, Pinole, CA (US); Eduardo Saiz, Berkeley, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/025,668

(22) Filed: Feb. 4, 2008

(51) Int. Cl. A61C 13/00; A61K 6/08

(52) U.S. Cl. 433/217.1; 523/116

(57) ABSTRACT

Provided are methods and compositions relating to a dental bonding composition useful in bonding a dental resin composite.

### BACKGROUND

The restoration of destroyed or decayed tooth structures can be achieved through the use of various materials including dental amalgams, composite resins, porcelain, or gold. One method used involves the sequential application of a dental adhesive followed by a dental restorative material to the affected tooth structure. Often the affected tooth structure is pretreated to improve the bonding of the adhesive to the dentin or the enamel of the affected tooth structure. For example, the bonding process may include three steps: (1) etching with an inorganic or organic acid to remove surface contaminants and to partially demineralize the dentin matrix; (2) priming with a monomer that can penetrate the collagen-rich network that remains after the etching step; and (3) application of an adhesive resin. The adhesive resin is typically cured to bond to a dental resin composite.

### SUMMARY

The disclosure provides methods and compositions relating to a dental bonding composition useful in bonding a dental resin composite.

Accordingly, the present disclosure provides methods for preparing a tooth for bonding to a dental resin composite. In certain embodiments, such methods include: applying an etching composition comprising an etchant to a tooth to produce an etched dentin surface; applying a priming composition comprising a primer to the etched dentin surface; applying an adhesive composition comprising a resin-based adhesive to the etched and primed dentin surface, where at least one of the adhesive composition or the priming composition includes a bioactive glass substantially lacking silanol groups and a non-aqueous solvent, and where the priming composition and the adhesive composition are optionally combined. Such methods can provide for formation of an adhesive layer and a hybrid layer, where the hybrid layer comprises dentin and the dental bonding composition.

In certain embodiments, such methods include that the primer of the priming composition is a self-etching primer, and the etching composition is optionally not applied in a separate step. In certain embodiments, such methods include that the resin-based adhesive is a self-etching adhesive, and the etching composition and the priming composition are optionally not applied in separate steps. In certain embodiments, such methods include that the adhesive composition comprises the primer, and the priming composition is optionally not applied in a separate step. In certain embodiments, such methods include that the bioactive glass is present at about 0.5% to 10% by weight percentage of the priming composition or the adhesive composition.

Also provided are methods for preparing a tooth for bonding to a dental resin composite. In certain embodiments, such methods include: applying an etching composition comprising an etchant to a tooth to produce an etched dentin surface; applying a bioactive glass composition comprising a bioactive glass substantially lacking silanol groups and a non-aqueous solvent; applying a priming composition comprising a primer to the etched dentin surface; and applying an adhesive composition comprising a resin-based adhesive to the etched and primed dentin surface. Such methods can provide for formation of an adhesive layer and a hybrid layer, where the hybrid layer comprises dentin and the dental bonding composition. In certain embodiments, such methods include that the bioactive glass is present at about 0.5% to 40% by weight percentage of the bioactive glass composition.

Also provided are dental bonding compositions that find use in the subject methods. Such dental bonding compositions may include a bioactive glass substantially lacking silanol groups; at least one of a resin-based adhesive and a primer; and a non-aqueous solvent comprising an alcohol. In exemplary embodiments, the bioactive glass has the following approximate composition by weight percentage: SiO₂ (44%), Na₂O (23%), CaO (10%), MgO (4.5%), P₂O₅ (6%), and CaF₂ (12.5%). In further embodiments, the bioactive glass has an average particle size of 1 μm or less. In still further embodiments, the dental bonding composition has about 0.5% to 1% by weight of said bioactive glass. In an exemplary embodiment, the alcohol solvent is ethanol.

Also provided are methods for making an adhesive composition for bonding a dental resin composite to dentin. Such methods may include mixing in a non-aqueous solvent a bioactive glass substantially lacking silanol groups and at least one of a resin-based adhesive and a primer. In certain embodiments, such methods include that the non-aqueous solvent is an alcohol, such as but not limited to ethanol.

Also provided are kits containing the dental bonding composition for use in the subject methods. In certain embodiments, the kits may include a dental bonding composition that comprises a bioactive glass substantially lacking silanol groups; at least one of a resin-based adhesive and a primer; and a non-aqueous solvent comprising an alcohol; optionally, an etching composition; and optionally, a resin-based composite suitable for use with the dental bonding composition. In further embodiments, the kit may include that the dental bonding composition is provided as separate components, where a first component comprises the bioactive glass in the non-aqueous solvent, where the first component is provided in a first container; and a second component comprises at least one of the resin-based adhesive and the primer, where the second component is provided in a second container. In further embodiments, the kit may include that the dental bonding composition is provided as separate components, where a first component comprises the bioactive glass, the primer, the resin-based adhesive, and the non-aqueous solvent, where the first component is provided in a first container. In still further embodiments, the kit may include that the dental bonding composition is provided as separate components, where a first component comprises the bioactive glass, the primer, and the non-aqueous solvent, where the first component is provided in a first container; and a second component comprises the resin-based adhesive.

### BRIEF DESCRIPTION OF THE FIGURES

FIG. 1 shows scanning electron microscopy (SEM) micrographs of cryofractured teeth at 2000× magnification: (a) vacuum-impregnated with Bioglass 45S5, (b) negative control. An apparently normal hybrid layer has formed in the Bioglass 45S5-treated teeth. “A” is adhesive; “H” is hybrid layer; and “D” is dentin.

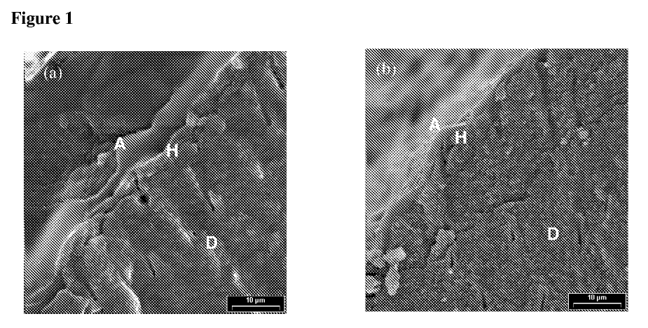

FIG. 2 shows SEM micrographs and energy dispersive x-ray (EDX) maps of teeth that have been bonded and soaked in simulated body fluid (SBF) for one week (Panels (i)-(iv)). Glass-treated samples (Panels (i) and (ii)) show little to no leakage after being soaked in SBF for one week, whereas negative controls (Panels (iii) and (iv)) exhibit extensive leakage. Bioactive glass-treated samples show little to no leakage after being soaked in SBF for two weeks (data not shown). Bioactive glass-treated samples that have not been exposed to simulated body fluid exhibit leakage between the adhesive and dentin (data not shown). Silicon was present in high concentrations in the composite, in reduced concentrations in the adhesive, and in further reduced concentrations in the dentin. (data not shown). EDX maps show the concentration of silver, as represented by white pixels. (C=composite, A=adhesive, D=dentin).

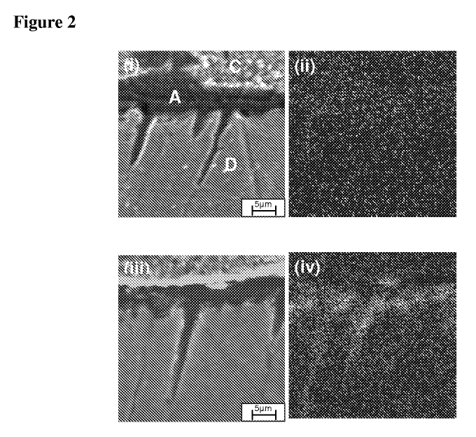

### DEFINITIONS

The term “dentin” as used herein refers to a calcified tissue of the body that is one of the major components of teeth. Dentin is usually covered by enamel, which forms the outer surface of the tooth. Dentin is a porous matrix composed of up to 70% hydroxyapatite. Dentin has microscopic channels, called dentinal tubules, which span the thickness of the dentin. Dentinal tubules taper in diameter from the inner to the outermost surface of the dentin, having a diameter of about 2.5 μm near the inner surface of the dentin, about 1.2 μm in the middle of the dentin, and about 900 nm near the outer surface of the dentin. In addition, dentine tubules are surrounded by collagen fibers, which form an extensive collagen network.

The term “etch” or “etching” as used herein means applying an acid to the surface of a tooth to partially dissolve the apatite and produce irregularities in the surface of dentin.

The term “prime” or “priming” as used herein means applying a compound to an acid-etched surface of a tooth to facilitate stabilization of the collagen network in the demineralized dentin, such as may result from an etching process. Dental primers also include self-etching primers, which achieve the steps of etching and priming in a single application step. Self-etching primers may include acidic monomers. Thus, reference to an “etched and primed surface” is meant to encompass etching and priming in separate steps or in a single step.

The terms “dental resin adhesive”, “dental adhesive”, “adhesive”, “adhesive resin”, or “resin-based adhesive” as used herein refer to compounds useful in facilitating a bond between a dental resin composite to a tooth. Adhesives may include a mixture of monomeric molecules that polymerize upon curing. Adhesives may be cured using light or a catalyst. Dental adhesives also include self-etching adhesives. A self-etching adhesive is an adhesive that contains compounds (i.e., a self-etching primer, such as an acidic monomer, and an adhesive) that achieve the steps of etching, priming, and bonding in a single application step.

Adhesives can be “unfilled”, wherein the adhesive is composed of compounds that actively participate in the polymerization and bonding process. Adhesives can be “filled”, wherein the adhesive contains compounds that do not participate in the polymerization and bonding process. Examples of fillers include, but are not limited to, silica powder, glass beads, aluminum oxide powder, and quartz powder.

The term “hybrid layer” as used herein refers to a layer between an adhesive composition disclosed herein and dentin that includes a molecular-level mixture of the adhesive and dentin. The hybrid layer can be created by diffusion of the adhesive resin into dentin that has been prepared by, for example, acid-etching of a dentin surface.

The terms “dental resin composite”, “dental composite” or “composite” as used herein refer to a type of restorative material used in dentistry. Dental resin composites are typically composed of a resin-based matrix, exemplified by, but not limited to, bisphenol α-glycidyl methacrylate (Bis-GMA), triethylene glycol dimethacrylate (TEGDMA), urethane dimethacrylate (UDMA), bisphenol α-polyetheylene glycol diether dimethacrylate (Bis-EMA(6)). Dental resin composites may also include an inorganic filler such as silicon dioxide (silica), or various glasses.

The terms “substantially lacks” or “substantially lacking” as used herein refer to a compound that is at least about 60% free, or about 75% free, or about 90-95% free from a component. For example, “substantially lacking silanol groups” refers to a compound that is at least about 60% free, or about 75% free, or about 90-95% free of silanol groups.

The term “non-aqueous solvent” is meant to encompass solvents that do not contain water as a predominant component, and include solvents that contain, for example, less than 15% water by volume, less than 10% water by volume, less than 5% water by volume, less than 1% water by volume, and may contain no detectable water.

### DESCRIPTION OF EXEMPLARY EMBODIMENTS

Exemplary embodiments of the methods and compositions of the present disclosure are provided below. These include methods for preparing dentin for bonding to a dental resin composite, methods for forming an adhesive bond between a dental resin composite and dentin, methods for making an adhesive composition useful in such methods, adhesive compositions useful in such methods, and kits useful in such methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any element, including optional elements. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Dental bonding compositions of the present disclosure will be described first, followed by a detailed description of exemplary uses for the dental bonding compositions.

### Dental Bonding Compositions

A dental bonding composition for bonding a dental resin composite to dentin is provided. The term “dental bonding compositions” is intended to refer to any bioactive glass-containing composition that can find use in one or more steps of a dental bonding method according to the present disclosure. Thus dental bonding compositions can provide for activity as one or more of an etchant (as in etching of dentin), primer (to prime a dentin surface), adhesive (to facilitate bonding of a composite to dentin), or combinations thereof (e.g. a self-etching primer, self-etching adhesive, and the like). In the dental bonding compositions, a bioactive glass is provided so that the bioactive glass contacts the dentin.

Thus, dental bonding compositions include, but are not necessarily limited to, compositions comprising a suitable non-aqueous solvent (e.g. an alcohol such as ethanol) and bioactive glass (e.g. to be applied to an etched dentin surface); a primer (which may be a self-etching primer) and bioactive glass; a resin-based adhesive (which may be a self-etching adhesive) and bioactive glass; and combinations thereof (e.g. a composition comprising bioactive glass, an etchant, a primer and a resin-based adhesive; a composition comprising bioactive glass, a self-etching primer and a resin-based adhesive). The bioactive glass present in the dental bonding composition is generally a bioactive glass that substantially lacks silanol groups. Exemplary dental bonding compositions are described in more detail below.

In certain cases, the dental bonding composition may include a bioactive glass, a suitable non-aqueous solvent, and one or both of a resin-based adhesive and a primer. In some embodiments, the primer is a self-etching primer (e.g. an acidic monomer). In these cases, the dental bonding composition can comprise from about 0.5% to 10% by weight, about 0.5% to 5% by weight, or about 0.5% to 1% by weight of the bioactive glass. In cases where a higher weight % of the bioactive glass is described, suspension of the bioactive glass in the resin-based adhesive, primer or acidic monomer may be facilitated by use of a bioactive glass powder having a smaller average particle size (e.g. less than 1 μm average particle size).

The dental bonding composition can be provided as components to be combined prior to use. For example, the resin-based adhesive can provided as two components, with a first component comprising a first monomer and a second adhesive component comprising a second monomer. The bioactive glass is provided in combination with the first or second adhesive component. In these embodiments, the bioactive glass may be present at a weight % that results in a desired weight % of bioactive glass in the final resin-based adhesive following combination of the components. Thus the weight % of bioactive glass in the first or second adhesive component can be about twice that of the final resin-based adhesive. It should be noted that the first and second monomers may be the same or different.

In other embodiments, the dental bonding composition is provided as bioactive glass suspended in a suitable non-aqueous solvent (e.g. a slurry). In these embodiments, the dental bonding composition comprises about 5% by weight, 10% by weight, about 15% by weight, about 20% by weight, about 25% by weight, about 30% by weight, about 35% by weight, or about 40% by weight, or more of the bioactive glass. The amount of bioactive glass incorporated into the dental bonding composition can vary with the average particle size of the bioactive glass. Smaller average particle sizes (e.g. 1 μm or less) may allow for more bioactive glass to be suspended in the dental bonding composition mixture.

In certain cases, the bioactive glass used in the dental bonding composition is Bioglass 45S5. In other cases, the bioactive glass used in the dental bonding composition is F glass and has the following approximate composition by weight percentage: SiO₂ (44%), Na₂O (23%), CaO (10%), MgO (4.5%), P₂O₅ (6%), and CaF₂ (12.5%). In either case, the bioactive glass used in the dental bonding composition may have an average particle size of 1 μm or less.

The bioactive glass is dispersed in the dental bonding composition via a solvent. As discussed below, the water content of the solvent is selected so that reaction of the bioactive glass with water in the solvent is insignificant, and may be so low as to avoid such reaction.

The dental bonding composition can facilitate the inhibition of leakage of particulate materials and/or fluid from dentin or the oral environment treated with the dental bonding composition (and, where a dental composite is adhered, between a dental composite adhered to the dental bonding composition-treated surface). This feature can be due not only to the coverage provided at a previously exposed dentin surface (and the seal with the dental composite), but also by formation of hybrid layer between the outermost dental bonding composition-covered surface and the dentin. Inhibition of leakage can include inhibition of both microleakage and nanoleakage. Microleakage is the seepage of fluids, debris, and/or microorganisms (e.g. bacteria) into micrometer-sized gaps (approximately 10⁻⁶ m) between a dental restoration and a tooth. Nanoleakage is the seepage of fluids, debris, and/or microorganisms (e.g. bacteria) into nanometer-sized gaps (i.e., approximately 10⁻⁹ m) between a dental restoration and a tooth. Without being held to theory, the ability of bioactive glasses to promote the formation of apatite in aqueous environments that contain calcium and phosphate (e.g. saliva) can facilitate inhibition of leakage at the bonded interface through a mechanism of self-sealing due to the formation of apatite.

Bioactive glass can be incorporated into the dental bonding composition so that the presence of the bioactive glass does not decrease the shear bond strength of the adhesive bond as compared to the shear bond strength of an adhesive bond that contains no bioactive glass. In some embodiments, incorporation of a bioactive glass into the dental bonding composition increases the shear bond strength of the adhesive bond as compared to the shear bond strength of an adhesive bond that contains no bioactive glass.

Bioactive Glass Compounds

Bioactive glasses elicit a series of chemical reactions when they are brought into contact an aqueous environment that contains calcium and phosphate, such as bone or tissue, leading to the formation of carbonated hydroxyapatite (HCA), which is similar to the mineral that forms teeth. The formation of HCA creates a bond between the bioactive glass and the dentin. The bond may be a mechanical bond and/or due to a chemical interaction between the bioactive glass and the dentin, in which the forming HCA bonds to specific amino acids within the collagen matrix of the dentin.

Bioactive glasses may contain but are not limited to silicon dioxide (SiO₂), sodium oxide (Na₂O), calcium oxide (CaO), magnesium oxide (MgO), phosphorous pentoxide (P₂O₅), and calcium fluoride (CaF₂). Other components may be added such as boron, magnesium, aluminum, iron, titanium, fluorine, and silver. The addition of fluorine to bioactive glass can be rationalized for the following reasons: firstly, that the rate of apatite formation at the glass surface can be enhanced; and secondly, that the apatite formed will be less vulnerable to acid attack, both of which are desirable in bioactive glass to be utilized in dental applications. Magnesium may also be added to bioactive glass, as it has been shown to slow down the rate of apatite precipitation, thus leading to more controlled mineralization.

Bioactive glass for use in the compositions and methods disclosed herein generally are characterized by having predominantly more silicon dioxide (SiO₂) groups than silanol (H₃SiOH) groups. In general, this can be achieved by avoiding contact of bioactive glass with water and/or hydrogen atoms, thereby avoiding production of silanol in the bioactive glass composition (e.g. as by the reaction Si—O—Na⁺+H⁺+OH⁻→Si—OH⁺+Na⁺+OH⁻). Accordingly, bioactive glass can be described as having, as a total of SiO₂ and H₃SiOH groups, greater than 50% SiO₂ groups, greater than 60% SiO₂ groups, greater than 75% SiO₂ groups, greater than 85% SiO₂ groups, greater than 90% SiO₂ groups, greater than 95% SiO₂ groups, and can be described as having, as a total of SiO₂ and H₃SiOH groups, at least 55% SiO₂ groups, at least 65% SiO₂ groups, at least 80% SiO₂ groups, at least 90% SiO₂ groups or more. In some embodiments, the bioactive glass is characterized as “substantially lacking silanol groups”, which as set out above refers to a bioactive glass that is at least about 60% free, or about 75% free, or about 90-95% free of silanol groups.

Bioactive glass compositions can be maintained in a relative “dehydrated” state prior to use. The term “dry” as used herein can refer to compositions kept at ambient conditions, for example at standard temperature, pressure, and humidity. It should be noted, however, that a “dry” compound can be provided in a substantially non-aqueous solvent (e.g. an alcohol). Thus, for example, slurries of bioactive glass powder in suitable non-aqueous solvent (e.g. an alcohol (e.g. ethanol)) are encompassed within the meaning of a “dry” bioactive glass composition. In some cases, the bioactive glass compositions may be stored in suitable packaging to keep the bioactive glass compositions dry (e.g. a sealed container).

Bioactive glass for use in the compositions and methods disclosed herein can be selected so as to have an average particle size that allows the bioactive glass particles to penetrate into the lumens of dentinal tubules. Dentinal tubules generally have diameters of approximately 0.8 μm to 1 μm before etching, and may have diameters of approximately 1 μm or greater after etching. Accordingly, suitable bioactive glass compositions for incorporation in the adhesive compositions of the present disclosure include those having an average particle size of 1 μm or less. Bioactive glass compositions can have a particle size distribution of at least 25%, at least 50%, at least 75%, at least 85% or more (e.g. 99%) of the particles are of an average particle size of 1 μm or less. However, bioactive glass containing particles having average particle sizes of less than 5 μm may also find use in the adhesive compositions of the present disclosure.

Exemplary bioactive glasses are described below.

Bioglass 45S5

Bioglass formulation 45S5, or Bioglass 45S5, is a bioactive glass that is composed of 49.5% SiO₂, 17.0% NaO, 26.9% CaO, and 6.6% P₂O₅, in weight %. In certain embodiments, Bioglass 45S5 is a powder, and can be provided as a dry powder. In these embodiments, the Bioglass 45S5 powder is composed of nanoparticles with an average particle size of 1 μm or less. An average particle size of 1 μm or less allows the bioactive glass powder to penetrate into the lumens of dentinal tubules, which have diameters of approximately 0.8 μm to 1 μm before etching and may have diameters of approximately 1 μm or more after etching. In addition, an average particle size of 1 μm or less can facilitate penetration of the bioactive glass into the partially etched intertubular dentin, which has openings of less than 1 μm. Bioglass 45S5 powder may be prepared by methods known to those of skill in the art, including but not limited to planetary ball milling of Bioglass 45S5 glass chips.

F Glass

F glass is a bioactive glass that has the following approximate composition by weight percentage: SiO₂ (44%), Na₂O (23%), CaO (10%), MgO (4.5%), P₂O₅ (6%), and CaF₂ (12.5%). In certain embodiments, F glass is a powder, and can be provided as a dry powder. In these embodiments, the F glass powder may be composed of nanoparticles with an average particle size of 1 μm or less. An average particle size of 1 μm or less allows the bioactive glass powder to penetrate into the lumens of dentinal tubules, which have diameters of approximately 0.8 to 1 μm before etching and may have diameters of approximately 1 μm or more after etching. In addition, an average particle size of 1 μm or less may allow the bioactive glass to penetrate into the partially etched intertubular dentin, which has openings of less than 1 μm. F glass powder may be prepared by methods known to those of skill in the art, including but not limited to planetary ball milling of chips of F glass.

Solvents

The solvent used to disperse the bioactive glass in the dental bonding composition can be any suitable solvent available in the art. As described above, bioactive glasses elicit a series of chemical reactions when they are brought into contact with tissue, or any aqueous environment that contains calcium and phosphate, leading to the formation of carbonated hydroxyapatite (HCA). Accordingly, the solvent can be described as a “non-aqueous solvent”, which refers to solvents that do not contain water as a predominant component, and include, for example, solvents that contain less than 10% water by volume, less than 5% water by volume, less than 1% water by volume, and may contain no detectable water. Such non-aqueous solvents, thus, have a water content that is sufficiently low to avoid reaction of the bioactive glass so as to significantly generate silanol groups. Thus, in some cases, the solvent for the adhesive composition contains less than 5% to less than 1% water, and can substantially lack water. In certain embodiments, the solvent is an alcohol. In these embodiments, the alcohol solvent may be ethanol, isopropyl alcohol, or any other suitable alcohol. In other cases, suitable solvents may include acetone. In some cases, the non-aqueous solvent is other than acetone.

The amount of solvent used in the dental bonding composition can vary according to the desired properties of the composition. For example, solvent can be added (or removed, e.g. by vacuum or evaporation) so as to provide a final dental bonding composition having a desired viscosity or consistency. For example, the composition can be flowable at ambient temperature, and may be of a consistency compatible with painting the composition onto the surface to be treated. Exemplary compositions can have the consistency of a fluid paste or gel. In general, the viscosity of the composition is compatible with its use so as to allow the composition to penetrate to a sufficient degree into the dentin matrix.

Resin-Based Adhesives

The resin-based adhesive can be any suitable resin-based adhesive available in the art. In some cases, the resin-based adhesive may include monomers that polymerize upon curing. In certain embodiments, the resin-based adhesive may be, but is not limited to, 2-hydroxyethylmethacrylate (HEMA), bisphenol α-glycidyl methacrylate (Bis-GMA), urethane dimethacrylate (UDMA), or triethylene glycol dimethacrylate (TEGDMA), or combinations thereof. In certain instances, the resin-based adhesive may be as described in any of the following U.S. Pat. No. 5,270,351; 5,348,988; 5,401,783; and 5,789,610, the relevant disclosures of which are incorporated by reference. It may be desirable to modify commercially available resin-based adhesives so as to deplete water from such prior to use, e.g. so as to minimize water that may come into contact with the bioactive glass. Alternatively or in addition, it may be desirable to combine such resin-based adhesives with the bioactive glass just prior to use, so as to minimize exposure of the bioactive glass to water prior to contacting with a dentin surface. Where the adhesive as provided commercially contains water, it may be desirable to treat the adhesive so as to deplete water prior to combination with the bioactive glass.

In some cases, the adhesive may be a self-etching adhesive. A self-etching adhesive is an adhesive that contains compounds (i.e., a self-etching primer, such as an acidic monomer, and an adhesive) that achieve the steps of etching, priming, and bonding in a single application.

Primers

The dental primer can be any suitable primer available in the art. Primers may contain molecules with hydrophilic groups that are capable of infiltrating and adhering to dentin, as well as hydrophobic groups that adhere to the adhesive resin. Dental primers may include, but are not limited to, 2-hydroxyethyl methacrylate (HEMA), hydroxyethyl trimellitate anhydride (4-META), and biphenyl dimethacrylate (BPDM). It may be desirable to modify commercially available primers so as to deplete water from such prior to use, e.g. so as to minimize water that may come into contact with the bioactive glass. Alternatively or in addition, it may be desirable to combine such primers with the bioactive glass just prior to use, so as to minimize exposure of the bioactive glass to water prior to contacting with a dentin surface. Where the primer as provided commercially contains water, it may be desirable to treat the primer so as to deplete water prior to combination with the bioactive glass.

In some cases, the primer may be a self-etching primer. A self-etching primer is a primer that includes, for example, an acidic monomer, such that the steps of etching and priming are achieved in a single application. Exemplary self-etching primers or adhesives include, but are not limited to, Clearfil SE Bond (Kuraray America (Japan)), Adaper Easy Bond (3M ESPE); Brush and Bond (Parkell Prod. Inc.), G Bond (GC America Inc); IBOND self etch (Kulzer Dental Division); and Optibond All in One (Kerr Mfg.).

Bioactive Glass-Containing Dental Bonding Compositions for Use in Preparation and Bonding Methods

As noted above, dental bonding compositions containing bioactive glass can include compounds to facilitate one or more steps of a dental bonding method according to the present disclosure. Thus, dental bonding compositions can provide for activity as one or more of an etchant (as in etching of dentin), primer (to prime a dentin surface), adhesive (to facilitate bonding of a composite to dentin), or combinations thereof (e.g. to provide for any combination (including all) steps of dental bonding methods). In embodiments of the dental bonding composition that do not contain a self-etching primer, a separate etchant is used to provide for demineralization of the dentin. In these embodiments, the dental etchant may be an inorganic or organic acid, such as but not limited to phosphoric acid, maleic acid, or citric acid.

For example, in one embodiment, the dental bonding composition can include a primer, a bioactive glass, and a non-aqueous solvent. In these embodiments, the dental bonding composition finds use in methods that include the sequential steps of: etching; contacting the etched surface of dentin with the dental bonding composition, such that the etched surface of dentin is primed by the dental bonding composition; and contacting the etched and primed surface of dentin with an adhesive. The primer can be a self-etching primer (e.g. an acidic monomer). Thus, for example, the dental bonding composition includes a self-etching primer, a bioactive glass, and a non-aqueous solvent. In these embodiments, the dental bonding composition may find use in methods that include contacting a tooth with the dental bonding composition, such that the steps of etching and priming are achieved in a single application. In these embodiments, the method may further include contacting the etched and primed surface of dentin with an adhesive.

In other embodiments, the dental bonding composition includes a primer, a bioactive glass, an adhesive, and a non-aqueous solvent. In these embodiments, the dental bonding composition may find use in methods that include contacting a tooth with an etchant to provide an etched dentin surface, and contacting the etched dentin with the dental bonding composition, such that the steps of priming and bonding are achieved in a single application step.

In other embodiments, the dental bonding composition includes a self-etching adhesive and a non-aqueous solvent, where the self-etching adhesive may include a self-etching primer, a bioactive glass, an adhesive, and a non-aqueous solvent. In these embodiments, the dental bonding composition may find use in methods that include contacting a tooth with the dental bonding composition, such that the steps of etching, priming, and bonding are achieved in a single application step.

In other embodiments, the dental bonding composition includes a suspension (e.g. slurry) of a bioactive glass in a non-aqueous solvent. In these embodiments, the dental bonding composition may find use in methods that include the steps of: etching; contacting the etched surface of dentin with the dental bonding composition; priming; and bonding.

In any of the above embodiments, the solvent may be an alcohol, such as but not limited to ethanol.

### Methods of Making

Methods for making a dental bonding composition for bonding a dental resin composite to dentin are also provided. In general, such methods can involve combining a bioactive glass as described above with a suitable non-aqueous solvent (e.g. to generate a slurry), then combining this composition with a resin-based adhesive, primer or acidic monomer.

In certain cases, the method includes mixing a bioactive glass as described above with a resin-based adhesive, primer or acidic monomer, where the resin-based adhesive, primer or acidic monomer is provided in a non-aqueous solvent (e.g. acetone, or an alcohol, such as but not limited to ethanol) prior to combining with the bioactive glass. Thus, for example, such methods can alternatively involve combining a resin-based adhesive, primer or acidic monomer with a non-aqueous solvent, then combining this composition with a bioactive glass.

The types and amounts of bioactive glass, as well as the types of solvents and resin-based adhesives, primers and acidic monomers can be those as exemplified herein.

The methods for production of a dental bonding composition contemplate production of intermediate compositions, which can later be combined to form the final dental bonding composition. For example, the bioactive glass can be provided in a non-aqueous solvent in a first container and a resin-based adhesive, primer or acidic monomer can be provided in a second container. All or part of the contents of the two containers can then be combined prior to use according to directions which can be optionally provided with the containers. In another example, the bioactive glass can be provided as a powder in a first container and a resin-based adhesive, primer or acidic monomer in a non-aqueous solvent provided in a second container. All or part of the contents of the two containers can then be combined prior to use, according to directions which can be optionally packaged with the containers.

### Methods of Use

The dental bonding compositions described herein may be used in dental restoration procedures to form an adhesive bond between a dental resin composite and a tooth. Generally, the steps involved for bonding a dental resin composite to a tooth include etching the tooth, priming the etched surface of the tooth, and bonding the dental resin composite to the tooth with the dental bonding composition described herein.

Effectively bonding resin to a tooth may require preparation of the tooth prior to bonding due to the hydrophilic nature of dental tissue, such as dentin, in contrast with the largely hydrophobic nature of some polymeric resins. To facilitate the formation of a mechanical bond between the resin and dentin, the steps described herein of etching, priming, and bonding may be used.

Etching involves applying an acid to the surface of a tooth to superficially demineralize the apatite of dentin. Etching may also remove surface contaminants, also known as the “smear layer” on the surface of dentin, Etching dentin exposes a layer of collagen fibers. Removal of the smear layer also exposes the dentinal tubules. Etching increases the surface area available for bonding and facilitates penetration of an adhesive into the porosities in the dentin revealed by the etching procedure. Penetration of the adhesive into the dentin matrix forms a hybrid layer that is composed of the dentin and the adhesive. This facilitates the formation of a mechanical bond after curing the adhesive. Dental etchants may be an inorganic or organic acid. such as but not limited to phosphoric acid, maleic acid, citric acid, or self-etching acidic monomers.

After etching, the dentin may be primed. Priming the acid-etched surface of dentin facilitates maintenance of the structure of the demineralized dentinal collagen, thereby promoting enhanced diffusion of adhesive resins into the demineralized dentin. Primers may contain molecules with hydrophilic groups that are capable of infiltrating and adhering to dentin, as well as hydrophobic groups that adhere to the adhesive resin. Dental primers may be a self-etching primer (e.g. an acidic monomer) as described herein. Dental primers may include, but are not limited to, 2-hydroxyethyl methacrylate (HEMA), hydroxyethyl trimellitate anhydride (4-META), and biphenyl dimethacrylate (BPDM).

The subsequent step of bonding the dental resin composite to the dentin with the dental bonding composition may include contacting the etched and/or primed surface of dentin with the dental bonding composition, and contacting the dental bonding composition with the dental resin composite. Prior to contacting the etched and/or primed surface of dentin with the dental bonding composition, the dental bonding composition may be made as described herein. In some cases, the dental bonding composition may be made in advance and stored until used. The dental bonding composition may be stored in a sealed container, such that during storage, the dental bonding composition remains substantially free of water.

As noted above, the dental binding compositions can provide for activity as one or more of an etchant (as in etching of dentin), primer (to prime a dentin surface), adhesive (to facilitate bonding of a composite to dentin), or combinations thereof (e.g. to provide for any combination (including all) steps of dental bonding methods). Accordingly, the dental bonding method can involve various steps according to the dental bonding compositions used. For example, the dental bonding composition can be applied to an etched dentin surface, and the dental bonding composition can provide for one or more of application of bioactive glass, primer, and adhesive. In another example, the dental bonding composition can be applied to an etched and primed dentin surface, and the dental bonding composition can provide for application of the adhesive. In another example, the dental bonding composition can provide for both etching and adhesive application, or both etching and primer application. Exemplary methods are described below.

Sequential and Separate Etching, Priming, and Adhesive Treatment

For example, the method can include the sequential and separate steps of etching, priming and bonding. In these embodiments, the dentin is first etched with a dental etchant, as described herein. Then, the etched dentin is contacted with a dental bonding composition, where the dental bonding composition includes a primer, a bioactive glass, and a solvent. In these embodiments, the solvent may be an alcohol, such as but not limited to ethanol. Optionally, the solvent can be evaporated away from the primed dentin (e.g. gently blow-drying). The primer can be polymerized by a suitable method, for example, by free radical polymerization using catalysts or blue light (i.e., approximately 540 nm) according to procedures common and well known by those skilled in the art.

Next, the etched and primed surface of dentin is contacted with an adhesive. Upon application of the adhesive, the adhesive penetrates into the dentin, forming a hybrid layer between the adhesive and the dentin. The hybrid layer of the resin-reinforced dentin improves bond strength between the adhesive and dentin, sealing the surface against leakage and imparting a high degree of acid resistance. The contacting of the adhesive to the dentin also forms an adhesive layer overlaying the hybrid layer.

In certain embodiments, after contacting the etched and primed surface of dentin with the adhesive, the adhesive may be cured. Curing the adhesive results in the polymerization of the monomers of the adhesive into cross-linked polymers, which hardens the adhesive, thereby bonding the adhesive composition to the tooth. In some cases, the adhesive contains photo initiators that allow the adhesive to be cured by exposing the adhesive to light. In these cases, the light may be blue light (i.e., approximately 540 nm). In other embodiments, curing of the adhesive may be initiated by mixing the adhesive with a catalyst immediately prior to use. Any suitable catalyst available in the art may be used. In certain cases, the catalyst may be an organic peroxide, such as but not limited to benzoyl peroxide.

As described herein, contacting the adhesive to the etched and primed surface of dentin also forms an adhesive layer overlaying the hybrid layer. In certain embodiments, the resulting adhesive layer may be contacted with a dental resin composite. After placement, the dental resin composite may be cured, thereby bonding the dental resin composite to the tooth. In some cases, curing may be achieved by exposing the dental resin composite to light, such as blue light (i.e., approximately 540 nm). In other cases, the curing process may be initiated by mixing the dental resin composite with a catalyst. In certain cases, the catalyst may be an organic peroxide, such as but not limited to benzoyl peroxide.

Combined Priming and Adhesive Treatment

In other embodiments, the method provides for priming and bonding by application of a single dental bonding composition. In these embodiments, the method includes contacting a tooth with an etchant to provide an etched surface of the dentin, and contacting the etched surface of the dentin with a dental bonding composition, where the dental bonding composition includes a primer, a bioactive glass, a resin-based adhesive, and a non-aqueous solvent. In these embodiments, the dental etchant may be an inorganic or organic acid, such as but not limited to phosphoric acid, maleic acid, or citric acid. In these embodiments, the solvent may be an alcohol, such as but not limited to ethanol. In this method, upon application of the dental bonding composition, the etched dentin is primed by the primer and the dentin matrix is penetrated by the bioactive glass and the adhesive, in a single application. The adhesive may be cured as described herein. In some cases, the method also includes contacting the adhesive layer with a dental resin composite such that the dental resin composite is adhered to the dentin.

Combined Etching and Priming Treatment

In other embodiments, the method provides for etching and priming by application of a single dental bonding composition, followed by application of an adhesive. In these embodiments, a tooth is contacted with a dental bonding composition, where the dental bonding composition includes a self-etching primer, a bioactive glass, and a solvent. In these embodiments, the solvent may be an alcohol, such as but not limited to ethanol. In these embodiments, the self-etching primer may be an acidic monomer as described herein. In this method, upon application of the dental bonding composition, the tooth is etched by the self-etching primer to expose the dentin matrix and the etched dentin matrix is penetrated by the self-etching primer and the bioactive glass, in a single application step. Subsequently, the etched and primed surface of dentin is contacted with an adhesive. The adhesive may be cured as described herein. In some cases, the method also includes contacting the adhesive layer with a dental resin composite such that the dental resin composite is adhered to the dentin.

Combined Etching, Priming, and Adhesive Treatment

In other embodiments, the dental bonding composition can include a self-etching adhesive. As described herein, the self-etching adhesive may include a self-etching primer (e.g. an acidic monomer), a bioactive glass, an adhesive, and a solvent. In these embodiments, the solvent may be an alcohol, such as but not limited to ethanol. In these embodiments, the self-etching primer may be an acidic monomer as described herein. In this method, upon application of the dental bonding composition, the dental bonding composition dissolves the smear layer, demineralizes the dentin to expose the dentin matrix, and primes the etched dentin surface. In addition, the bioactive glass and adhesive penetrate the demineralized dentin to form the hybrid layer, in a single application. Subsequently, the dental bonding composition may be cured as described herein. In some cases, the method also includes contacting the adhesive layer with a dental resin composite such that the dental resin composite is adhered to the dentin.

Application of Bioactive Glass-Containing Suspension

In certain embodiments, the method includes the steps of etching, contacting the etched surface of dentin with a suspension of a bioactive glass in a non-aqueous solvent, priming, and bonding. In these embodiments, the dentin is first etched with a dental etchant, as described herein. In these embodiments, the dental etchant may be an inorganic or organic acid, such as but not limited to phosphoric acid, maleic acid, or citric acid. Then, the etched dentin is contacted with a suspension of a bioactive glass (e.g. a slurry) in a non-aqueous solvent. In some cases, the non-aqueous solvent may be an alcohol, such as but not limited to ethanol. Subsequently, the tooth with bioactive glass may be contacted with a dental primer, which is then cured. Then, the primed surface of the dentin may be contacted with an adhesive. The adhesive may be cured as described herein. In some cases, the method also includes contacting the adhesive layer with a dental resin composite such that the dental resin composite is adhered to the dentin.

### Kits

Also of interest are kits for use in practicing certain embodiments of the invention. The components of the kits can be adapted for use in any of the various methods described herein.

In certain embodiments, the kits include a dental bonding composition of the present disclosure, which may be provided for immediate use, or may be provided as separate components to be combined to form the dental bonding composition. The dental bonding composition can be provided in a container, e.g. that can be adapted to facilitate application to an etched and/or primed dentin surface. For example, the container can be in the form of a needleless syringe, with a plunger to provide for application of the contents of the container to a etched and/or primed dentin surface.

For example, where the dental bonding composition is provided as separate components (e.g. as described above in the context of methods of making the dental bonding composition), the kit can include a first container containing a bioactive glass in a non-aqueous solvent, and a second container containing a resin-based adhesive, primer or acidic monomer. In another example, the kit can include a first container containing a bioactive glass (e.g. as a powder) and a second container containing a resin-based adhesive, primer or acidic monomer in a non-aqueous solvent. In certain embodiments, the kit can include a first container containing a bioactive glass and a first resin-based adhesive, primer or acidic monomer in a non-aqueous solvent, and a second container containing a second resin-based adhesive, primer or acidic monomer, which allows for mixing of all or part of the contents of the first container with all or part of the contents of the second container prior to application to a tooth. The first and second resin-based adhesives can have the same or different composition, and in some embodiments will differ in monomer composition.

Optionally, the kit can include instructions for combining all or a portion of the first and second containers to provide a dental bonding composition. provided in a second container; all or part of the contents of the two containers can then be combined prior to use, according to directions which can be optionally provided with the kit. In one embodiment, where the dental bonding composition is provided as separate components to be combined prior to use, the two components are maintained in separate containers that are not in fluid communication, but that are separated by a frangible or removable wall, which can be broken or removed to facilitate mixing of the two components prior to use.

The kits can optionally include additional components. For example, the kits can include one or both of an etchant and a primer compatible for use with the dental bonding composition. Such additional components can be provided in additional containers as may be desired.

In certain embodiments, the kit may include an etchant, a dental bonding composition, and an adhesive, where the etchant, the dental bonding composition, and the adhesive are provided in separate containers. In these embodiments, the dental bonding composition may include a primer, a bioactive glass, and a solvent.

In other embodiments, the kit may include an etchant and dental bonding composition, where the etchant and the dental bonding composition are provided in separate containers. In these embodiments, the dental bonding composition may include a primer, a bioactive glass, an adhesive, and a solvent.

In other embodiments, the kit may include a dental bonding composition provided in a single container. In some cases, the dental bonding composition may include a self-etching adhesive. In these cases, the self-etching adhesive may include a self-etching primer (e.g. an acidic monomer), a bioactive glass, an adhesive, and a solvent.

In other embodiments, the kit may include a dental bonding composition and an adhesive provided in separate containers. In these embodiments, the dental bonding composition may include a self-etching primer (e.g. an acidic monomer), a bioactive glass, and a solvent.

In other embodiments, the kit may include an etchant, a slurry of a bioactive glass in a solvent, a primer, and an adhesive, where the etchant, the slurry, the primer, and the adhesive are provided in separate containers.

In certain embodiments, the kits will further include instructions for practicing the subject methods or means for obtaining the same (e.g. a website URL directing the user to a webpage which provides the instructions), where these instructions may be printed on a substrate, where substrate may be one or more of: a package insert, the packaging, reagent containers and the like. In the subject kits, the one or more components are present in the same or different containers, as may be convenient or desirable.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Studies to test the inhibition of microleakage in resin-bonded dentin in the presence of Bioglass 45S5 were performed.

### Preparation of Tooth Samples

The occlusal enamel of six human third molars was removed using a belt sander with 240-grit silicon carbide paper, following which the roots were removed using a slow-speed saw (Isomet, Buehler Ltd., Lake Bluff, Ill.) with water coolant to produce tooth discs approximately 5 mm thick. The exposed dentin was polished with 320-grit silicon carbide paper. The discs were mounted on open-ended tubes with hot glue, and then attached to a vacuum trap. The occlusal dentin was etched for 15 seconds with Scotchbond gel etchant (3M/ESPE, St. Paul, Minn.) and rinsed for a further 15 seconds with deionized water. A slurry of 20% or 40% (w/v) bioactive glass of the formulation 45S5 (Bioglass 45S5, SEM-COM, Toledo, Ohio) in ethanol was applied to the top of the sample. The average particle size of the ground bioactive glass powder was approximately 1 μm, and it was prepared by planetary ball milling of glass chips. The glass slurry was prepared with ethanol. Samples were also prepared using a slurry of 40% Al₂O₃ powder (particle size 1 μm; Buehler, Lake Bluff, Ill.) in water. Vacuum was applied at 530 mm Hg for one minute; the sample surface was kept moist by re-applying the slurry every few seconds. After removal from the vacuum, any excess solid was gently rinsed away with deionized water. Negative controls were vacuum-aspirated with water alone. The sample surface was then bonded with Single Bond (3M/ESPE, St. Paul, Minn.) adhesive and light-cured per manufacturer's instructions. The samples were cryofractured and mounted on aluminum stubs, or prepared for leakage studies, for which two coats of Filtek Z-250 Universal Restorative Composite (3M/ESPE, St. Paul, Minn.) were applied and cured per manufacturer's instructions.

### Silver Nitrate Leakage Study

After soaking in simulated body fluid for a specified length of time (t=0 hours, t=1 week, or t=2 weeks), three serial slabs were cut from the central region of each tooth sample with a slow-speed saw. Each slab was coated with nail polish to within approximately 1 mm of the bonded interface and soaked in 50% (w/v) silver nitrate for two hours without exposure to light. The samples were then transferred to developing solution for four hours under fluorescent lights, after which they were thoroughly rinsed with deionized water. The nail polish was removed with acetone, and the samples were polished through 1200 grit SiC, then mounted on aluminum stubs for SEM analysis.

Scanning electron microscopy and energy-dispersive x-ray analysis (SEM/EDX)

SEM (Topcon ISI ABT SX-40A, Milpitas, Calif.) visualization of the samples was executed in one of two ways: either in secondary electron mode for topographical information or in backscattered mode, using a charge-free anticontamination system (CFAS) and energy dispersive x-ray analysis for chemical information (Thermo-Noran Sigma2, Middleton, Wis.). Samples viewed in secondary electron mode (15 keV) were first sputter-coated with a 200 nm-thick layer of gold/palladium under argon atmosphere. Samples designated for EDX were not coated with gold/palladium. Their chemical composition was mapped (dwell time, 5600 μs; 128×128 pixels) with regard to the following elements: silicon, sodium, calcium, and phosphorus for cryofractured Bioglass 45S5-impregnated samples; and silicon, sodium, calcium, phosphorus, and silver for silver nitrate leakage studies.

### Methylene Blue Leakage Study

Tooth samples were soaked in simulated body fluid overnight, then thermocycled in water baths for 500 cycles in the following pattern: 5 seconds at 5° C., 20 seconds at 37° C., 5 seconds at 55° C., and 20 seconds at 37° C. After thermocycling, the samples were coated with nail polish on the tooth surface to within approximately 1 mm of the bonded interface and inverted in individual wells of a multi-well plate so that they were about two-thirds submerged in a 2% (w/v) methylene blue solution for 14 hours. They were then removed from the methylene blue and rinsed with tap water for 30 minutes. The samples were allowed to air-dry, and then the nail polish and superficial methylene blue was removed by polishing. Longitudinal slices approximately 1 mm thick were cut in an occlusal to apical direction from the center of each tooth, using a slow-speed saw and xylene as a coolant. The slices were allowed to air-dry and mounted on glass microscope slides, then examined under a light microscope by two independent scorers and ranked. The ranking methodology was determined as follows: teeth were visually divided in half, so each tooth received four scores. Teeth with no dye penetration were given a score of zero, while penetration into the outer third was scored as 1, the middle third was scored as 2, and the inner third was scored as 3. The non-parametric Mann-Whitney Rank Sum Test was used to analyze the results.

### Results

SEM images of the cryofractured samples showed that an apparently normal hybrid layer formed in the dentin that had been vacuum-infiltrated with Bioglass 45S5. The adhesive permeated etched dentin and entered the tubules to form resin tags, thus sealing the microparticulate matter into the dentin. The vacuum aspiration process did not seem to have a deleterious effect on the dentin tubules; they did not appear to be narrowed, collapsed, or altered in any manner.

Although neither glass nor Al₂O₃ particles were easily detected in the SEM images, EDX maps showed that particles were embedded in the dentin to a depth of approximately 5-10 μm below the surface. The EDX maps further suggested that the materials are present in both the adhesive and the dentin itself.

Silver nitrate leakage studies indicated that the presence of bioactive glass appeared to reduce leakage in comparison to negative controls. Bioglass 45S5-treated samples that were studied prior to soaking in simulated body fluid exhibited leakage similar to that of negative controls; however, after storage in SBF for one or two weeks, leakage was apparently reduced in the bioactive glass-treated samples.

In the case of the methylene blue leakage studies, a statistically significant difference between bioactive glass-treated teeth and negative controls (Mann-Whitney U=39.0, p=0.0005) was found. Although 12 teeth were prepared, 1 sample was lost; of the others, rankings were discarded if the interface was obscured by either residual enamel or by overhanging composite. In all, there were eighteen separate rankings for six bioactive glass-treated teeth, and fourteen separate rankings for five negative controls. The rankings from both scorers were pooled in order to evaluate the difference between treatment groups. There was no significant difference between scorers (p=0.85); there was, however, a significant difference between sides among the groups, which was more pronounced for the negative controls (p=0.084, bioactive glass-treated teeth; p=0.012, negative controls).

The above experiments showed that microparticles of bioactive glass are effectively incorporated into the resin-dentin bonding process. Once incorporated into the bonded dentin, the microparticles of bioactive glass inhibit leakage when samples are soaked in simulated body fluid.

### Example 2

Studies to test the inhibition of microleakage in resin-bonded dentin in the presence of either Bioglass 45S5 or F glass were performed.

### Glass Preparation

Bioactive glass of the formulation 45S5 (Bioglass 45S5, SEM-COM, Inc., Toledo, Ohio) in chip form was ground to an average particle size of approximately 1 μm by planetary ball milling.

A glass incorporating fluoride (“F glass”) was prepared using the following compounds, with weight percentages in parentheses: SiO₂ (44%), Na₂O (23%), CaO (10%), MgO (4.5%), P₂O₅ (6%), and CaF₂ (12.5%). F glass was prepared using the methods as described in Lopez-Esteban, S., et al., Bioactive glass coatings for orthopedic metallic implants, Journal of the European Ceramic Society 2003, 23(15), 2921-2930. The resulting glass was ground to an average particle size of approximately 1 μm.

### Glass Analysis: X-Ray Diffraction and Scanning Electron Microscopy (SEM)

Slabs of F glass were cut from the prepared glass bars using a diamond blade and ethanol coolant, then polished through an ascending series of silicon carbide grits to final dimensions of approximately 25 mm×24 mm×2 mm, also with ethanol coolant. The glass slabs were soaked in simulated body fluid for approximately 6 weeks. X-ray diffraction analysis was performed on a Siemens D5000 instrument with Cu radiation, 40 kv, 30 ma and diffracted beam monochromator; step scan 0.04° 2θ at 2 sec per step. The scan ranges were usually 24-55° 2θ; although 5-24° 2θ was also examined. The resulting spectra were compared with Joint Committee on Powder Diffraction Standards (JCPDS) files for hydroxyapatite (9-432) and brushite (9-0077). SEM (Field Emission Variable Pressure SEM Model S-4300SE/N, Hitachi Hi-Technologies, London, UK) was performed at an accelerating voltage of 15 kV to visualize the samples.

### Vacuum Infiltration and Bonding

The occlusal enamel of human third molars was removed using a belt sander with 240-grit silicon carbide paper, following which the roots were removed using a slow-speed saw (Isomet, Buehler Ltd., Lake Bluff, Ill.) with water coolant to produce tooth discs approximately 5 mm thick. The exposed dentin was polished with 320-grit silicon carbide paper. The resulting discs were mounted on open-ended tubes with hot glue, and then attached to a vacuum trap. The exposed occlusal dentin was etched for 15 seconds with Scotchbond gel etchant (3M/ESPE, St. Paul, Minn.) and rinsed for a further 15 seconds with deionized water. A slurry of 10% (w/v) bioactive glass of the formulation 45S5 (Bioglass 45S5, SEM-COM, Toledo, Ohio) or 10% F glass in ethanol, was applied to the top of each sample. Vacuum was applied at 530 mmHg for one minute; the sample surface was kept moist by re-applying the slurry every few seconds or as needed. After removal from the vacuum, any excess solid was gently rinsed away with deionized water. Negative controls were vacuum-aspirated with water alone. Samples were removed from their mountings and the etched surface was then bonded with Single Bond (3M/ESPE, St. Paul, Minn.) adhesive and light-cured per manufacturer's instructions. Two coats (each approximately 1 mm thick) of Filtek Z-250 Universal Restorative Composite (3M/ESPE, St. Paul, Minn.) were applied and light-cured per manufacturer's instructions. Samples were stored in simulated body fluid at 37° C. for one or two weeks prior to either cryofracturing or preparation for the silver nitrate leakage study. Additionally, one set of samples was prepared for the silver nitrate leakage study immediately after bonding.

### Silver Nitrate Leakage Study

After soaking in simulated body fluid for a specified length of time (t=0 hours, t=1 week, or t=2 weeks), three serial slabs were cut from the central region of each tooth sample with a slow-speed saw. Tooth samples were cryofractured and mounted for SEM analysis. Each slab was coated with nail polish to within approximately 1 mm of the bonded interface and soaked in 50% (w/v) silver nitrate for two hours without exposure to light. The samples were then transferred to developing solution for four hours under fluorescent lights, after which they were thoroughly rinsed with deionized water. The nail polish was removed by grinding with 240 grit silicon carbide paper, and the samples were polished through 1200 grit SiC, and then mounted on aluminum stubs for SEM analysis.

### Scanning Electron Microscopy and Energy-Dispersive X-Ray Analysis (SEM/EDX)

SEM (Topcon ISI ABT SX-40A, Milpitas, Calif.) visualization of the samples was executed in backscattered mode, using a charge-free anticontamination system (CFAS) and energy dispersive x-ray (EDX) analysis for chemical information (Thermo-Noran Sigma2, Middleton, Wis.). Their chemical composition was mapped (dwell time, 5600 μs; 256×256 pixels) with regard to the following elements: calcium, magnesium, phosphorus, silicon, silver, and sodium.

### Methylene Blue Leakage Study

Tooth samples were soaked in simulated body fluid overnight, then thermocycled in water baths for 500 cycles in the following pattern: 5 seconds at 5° C., 20 seconds at 37° C., 5 seconds at 55° C., and 20 seconds at 37° C. After thermocycling, the samples were coated with nail polish on the tooth surface to within approximately 1 mm of the bonded interface and inverted in individual wells of a multi-well plate so that they were about two-thirds submerged in a 2% (w/v) methylene blue solution for 14 hours. They were then removed from the methylene blue and rinsed with tap water for 30 minutes. The samples were allowed to air-dry, and then the nail polish and superficial methylene blue was removed by polishing. Longitudinal slices approximately 1 mm thick were cut in an occlusal to apical direction from the center of each tooth, using a slow-speed saw and xylene as a coolant. The slices were allowed to air-dry and mounted on glass microscope slides, then examined under a light microscope by two independent scorers and ranked. The ranking methodology was determined as follows: teeth were visually divided in half, and each scorer ranked each half. Thus, each tooth received four scores. Teeth with no dye penetration were given a score of zero, while penetration into the outer third was scored as 1, the middle third was scored as 2, and the inner third was scored as 3. If the interface was obscured by composite (“flash”), then that side was not included for analysis. The non-parametric Kruskal-Wallis Test was used to analyze the results where three groups were compared; the non-parametric Mann Whitney Rank Sum Test was used when only two variables were compared.

### Results

X-ray diffraction analysis of the glass that was produced demonstrated its ability to form apatite when placed in an aqueous environment that contains calcium and phosphate, indicating a potential for bioactivity. Additionally, SEM analysis of the samples showed crystal formation that is consistent with apatite.

Results from the silver nitrate leakage study showed a reduction in leakage in the samples that were treated with both the F glass and the 45S5, then soaked in SBF, when compared to the negative controls. Leakage was evident to a similar degree in all samples on which silver nitrate leakage assessment was performed immediately after bonding (no soaking in SBF at all). Cryofractured samples illustrate the formation of an apparently normal hybrid layer in the glass-treated teeth.

The methylene blue leakage test following thermocycling (see Table 1 for a summary of results) did not suggest a statistically significant difference between treatments (Kruskal-Wallis H=1.50, p=0.471). When analyzed with the Mann-Whitney Rank Sum Test, appropriate for comparison of only two variables, there was no significant difference between operators for any of the three groups (negative control, p=0.52; F glass, p=0.97; 45S5, p=0.98); nor was there a significant difference between sides for any of the three groups (negative control, p=0.70; F glass, p=0.24; 45S5, p=0.34). These non-parametric tests were appropriate given the ordinal ranking design of the experiment, and the lack of assumption of a normally distributed data set. However, a trend was suggested by the data in that there were more sides with “0” scores in each of the two glass-treated groups (F glass, 4 scores of 0; 45S5, 10 scores of 0) than in the negative controls (3 scores of 0).

The methylene blue leakage test results likely differed from the results obtained from either SEM visualization or EDX analysis of samples because the storage time in simulated body fluid may have been too short to show a difference between test samples and controls.

TABLE 1
Summary of bond strength data

| Negative Control | | | F glass | | | 45S5 | | |
|---|---|---|---|---|---|---|---|---|
| Sample | L | R | Sample | L | R | Sample | L | R |

Operator 1

| | | | | | | | | |
|---|---|---|---|---|---|---|---|---|
| A | 1 | 1 | G | 1 | N/A | N | 0 | 1 |
| B | 1 | 1 | H | 0 | 1 | P | 0 | 3 |
| C | 1 | 1 | J | 0 | 1 | Q | 1 | 0 |
| D | 3 | 3 | K | 1 | 1 | R | 1 | 0 |
| E | 1 | 0 | L | 3 | N/A | S | 3 | 3 |
| F | 2 | 2 | M | 1 | 2 | T | 0 | 1 |

Operator 2

| | | | | | | | | |
|---|---|---|---|---|---|---|---|---|
| A | 1 | 1 | G | 1 | N/A | N | 0 | 1 |
| B | 1 | 0 | H | 0 | 1 | P | 0 | 3 |
| C | 1 | 1 | J | 0 | 1 | Q | 1 | 0 |
| D | 1 | 3 | K | 1 | 1 | R | 1 | 0 |
| E | 1 | 0 | L | 3 | N/A | S | 3 | 3 |
| F | 2 | 2 | M | 1 | 2 | T | 0 | 1 |

The above experiments showed that microparticles of Bioglass 45S5 or F glass inhibit leakage when bonded teeth are stored in an aqueous calcium- and phosphate-containing environment.

### Example 3

Studies to test whether vacuum infiltration of Bioglass 45S5 or F glass into the surface of dentin inhibits leakage in resin-bonded dentin without causing deleterious effects on bond strength were performed.

### Glass Preparation

Bioactive glass of the formulation 45S5 (Bioglass 45S5, SEM-COM, Inc., Toledo, Ohio) in chip form was ground to an average particle size of approximately 1 μm by planetary ball milling.

A glass incorporating fluoride (“F glass”) was prepared using the following compounds, with weight percentages in parentheses: SiO₂ (44%), Na₂O (23%), CaO (10%), MgO (4.5%), P₂O₅ (6%), and CaF₂ (12.5%). The compounds were mixed in ethanol with a high-speed stirrer, dried at 80° C. for 12 hours, and then fired in air in a Pt crucible for 4 hours at 1400-1500° C. The melt was cast into a graphite mold to produce approximately 50×50×5 mm plates. The plates were annealed at 500° C. for 6 hours. The resulting glass was ground to an average particle size of approximately 1 μm.

### Vacuum Infiltration and Bonding

The occlusal enamel of human third molars was removed using a belt sander with 240-grit silicon carbide paper, following which the roots were removed using a slow-speed saw (Isomet, Buehler Ltd., Lake Bluff, Ill.) with water coolant to produce tooth discs approximately 5 mm thick. The exposed dentin was polished with 320-grit silicon carbide paper. The resulting discs were mounted on open-ended tubes with hot glue, and then attached to a vacuum trap. A strip of Mylar with a circular (3.2 mm diameter) window was affixed to the dentin surface. The exposed occlusal dentin was etched for 15 seconds with Scotchbond gel etchant (3M/ESPE, St. Paul, Minn.) and rinsed for a further 15 seconds with deionized water. A slurry of 10% (w/v) bioactive glass of the formulation 45S5 (Bioglass 45S5, SEM-COM, Toledo, Ohio) or 10% F glass in ethanol, was applied to the top of each sample. Vacuum was applied at 530 mm Hg for one minute; the sample surface was kept moist by re-applying the slurry every few seconds. After removal from the vacuum, any excess solid was gently rinsed away with deionized water. Negative controls were vacuum-aspirated with water alone. Samples were removed from their mountings and placed into shear plates for the Single Plane Lap Shear Test, as described in Watanabe, L. G., et al., Dentin shear strength: effects of tubule orientation and intratooth location, Dent Mater 1996, 12(2), 109-15. The sample surface was then bonded with Single Bond (3M/ESPE, St. Paul, Minn.) adhesive and light-cured per manufacturer's instructions. The remainder of the shear test apparatus was assembled, following which two coats of Filtek Z-250 Universal Restorative Composite (3M/ESPE, St. Paul, Minn.) were applied and light-cured per manufacturer's instructions. Samples were stored in deionized water at 37° C. overnight, for 8 days, or for 65 days.

Additionally, samples were prepared as above, but were stored in simulated body fluid for 15 days.

### Bond Strength Test

About 1 hour prior to testing, samples were removed from the storage medium. Excess water was removed from around the tooth, and dental stone (Tuff Rock, Talladium, Inc., Valencia, Calif.) was poured to anchor the sample in the device. Once the stone hardened, samples were tested to failure on a universal mechanical testing machine (Instron Model 1122, Canton, Mass.) at a crosshead speed of 5 mm/min. Means and standard deviations were calculated and statistical analysis was performed using one-way ANOVA at a significance level of p<0.05.

### Scanning Electron Microscopy (SEM)

After mechanical testing, samples were sectioned through the area of failure using a slow-speed saw (Isomet, Buehler, Ltd., Lake Bluff, Ill.) in the direction that force was applied. The cut surface was polished through ascending grits of silicon carbide paper (through 1200 grit), then with 6 μm, 3 μm, 1 μm and 0.25 μm diamond paste (Metadi Monocrystalline Diamond Suspension, Buehler, Ltd., Lake Bluff, Ill.). Samples were then mounted on aluminum stubs and sputter-coated with a 200 nm thick layer of gold/palladium in preparation for scanning electron microscopy. SEM (Field Emission Variable Pressure SEM Model S-4300SE/N, Hitachi Hi-Technologies, London, UK) was performed at an accelerating voltage of 20 kV.

### Results

Bond strength tests showed no significant difference between F glass-treated teeth, 45S5-treated teeth, and negative controls at all time periods for samples that had been stored in water (Table 2).

TABLE 2
Shear bond strengths of samples stored in water

| | Overnight | | 8 Days | | 65 days | |
|---|---|---|---|---|---|---|
| | | Mean ± SD | | Mean ± SD | | Mean ± SD |
| | N | (MPa) | N | (MPa) | N | (MPa) |
| Negative | 6 | 31.4 ± 11.1 | 6 | 38.6 ± 6.6 | 6 | 29.2 ± 11.7 |
| Control | | | | | | |
| F Glass | 6 | 41.9 ± 3.6 | 6 | 36.2 ± 6.0 | 6 | 38.6 ± 6.4 |
| 45S5 | 6 | 37.6 ± 7.3 | 6 | 40.8 ± 9.0 | 6 | 31.7 ± 14.2 |
| | | p = 0.10 | | p = 0.57 | | p = 0.35 |

While the general trend showed a decrease in bond strength over time, none of the losses was statistically significant: for the negative controls, p=0.27; for the F glass-treated samples, p=0.22; and for the 45S5-treated teeth, p=0.35. Similarly, there was no significant difference between groups that had been stored in simulated body fluid for 15 days (Table 3). By the end of the storage period, the SBF had changed color, from clear to blue; this effect was determined to be due to pigment leaching from the sample labels and probably did not affect the outcome of the bond strength tests.

TABLE 3
Shear bond strengths of samples stored in simulated body fluid

| | N | Mean ± SD (MPa) |
|---|---|---|
| Negative control | 6 | 35.2 ± 5.2 |
| F glass | 6 | 23.6 ± 12.1 |
| 45S5 | 6 | 33.4 ± 11.6 |
| | | p = 0.14 |

SEM observation revealed that samples of all types failed in a variety of modes across all time periods, in both types of storage medium. In each category, adhesive failure was observed, as well as cohesive failure of both dentin and composite. However, it did appear that cohesive failure, either of dentin or of composite, appeared to be much more pronounced in samples that had been treated with F glass than either negative controls or 45S5-treated samples, for both types of storage medium.

The above experiments showed that Bioglass 45S5 or F glass incorporated into the dentin bonding process does not cause deleterious effects on the strength of the bonds formed.

### Example 4

Evaluation of whether incorporation of Bioglass 45S5 or F glass into resin-based dental adhesives inhibits leakage in resin-bonded dentin without causing deleterious effects on bond strength was performed.

### Glass Preparation

Bioactive glass of the formulation 45S5 (Bioglass 45S5, SEM-COM, Inc., Toledo, Ohio) in chip form was ground to an average particle size of approximately 1 μm by planetary ball milling.

A glass incorporating fluoride (“F glass”) was prepared using the following compounds, with weight percentages in parentheses: SiO₂ (44%), Na₂O (23%), CaO (10%), MgO (4.5%), P₂O₅ (6%), and CaF₂ (12.5%). F glass was prepared using the methods as described in Lopez-Esteban, S., et al., Bioactive glass coatings for orthopedic metallic implants, Journal of the European Ceramic Society 2003, 23(15), 2921-2930. The resulting glass was ground to an average particle size of approximately 1 μm.

### Shear Bond Testing

Either 1% or 0.5% wt/wt of the bioactive glass of the formulation 45S5 or F glass was combined with One Step or Part A of All Bond 3 (Bisco, Inc., Schaumburg, Ill.). Shear bond testing was conducted using bonded human dentin specimens according to manufacturer's instructions (Ultradent method, R^{D}-022, Ultradent, Inc., South Jordan, Utah). The dentin was exposed by grinding, and the teeth were polished in a randomized figure-eight motion for 30s on moist 320 grit sandpaper, rinsed, and etched with UniEtch (Bisco, Inc., Schaumburg, Ill.) for 15s. Teeth were then rinsed, and 2 coats of adhesive (One Step; or All Bond 3 Primer B combined in a 1:1 ratio with part B, part C, or part D) were applied on moist dentin, air thinned, and light cured for 10s. Aelite composite (Bisco, Inc., Schaumburg, Ill.) was used to fabricate the post. Controls were prepared using the same procedure, only without inclusion of any glass in the adhesive. After fabrication, all specimens were stored for 2 hours at 37° C., after which they were tested until failure at a crosshead speed of 1 mm/min (Instron 4466 C1879). Results were analyzed using One-Way Analysis of Variance (ANOVA).

### Silver Nitrate Leakage Study and Scanning Electron Microscopy/Energy Dispersive X-Ray Analysis (SEM/EDX)

Tooth samples were cut in half in an occlusal to apical orientation. The occlusal dentin of each half was prepared using the Ultradent procedure (RD-021; Ultradent, Inc., South Jordan, Utah)); the etching and adhesive application procedures were the same as stated previously. Light Core Blue composite was applied and cured in 3-4 increments with a cure of 40s/increment. Controls were prepared using the same procedure, only without inclusion of any glass in the adhesive. Teeth were stored in water for 1 day. Three serial slabs were cut from the central region of each half-tooth with a slow-speed saw (Isomet, Buehler Ltd., Lake Bluff, Ill.). Each slab was coated with nail polish to within approximately 1 mm of the bonded interface and soaked in 50% (w/v) silver nitrate for two hours without exposure to light. The samples were then transferred to developing solution for four hours under fluorescent lights, after which they were thoroughly rinsed with deionized water. The nail polish was removed by grinding with 240 grit SiC paper, the samples were polished through 1200 grit SiC, and then mounted on aluminum stubs for SEM analysis.

### Scanning Electron Microscopy and Energy-Dispersive X-Ray Analysis (SEM/EDX)

SEM (Topcon ISI ABT SX-40A, Milpitas, Calif.) visualization of the samples was executed in backscattered mode at an accelerating voltage of 15 keV, using a charge-free anticontamination system (CFAS) and energy dispersive x-ray analysis for chemical information (Thermo-Noran Sigma2, Middleton, Wis.). Their chemical composition was mapped (dwell time, 5600 μs; 256×256 pixels) with regard to the following elements: calcium, magnesium, phosphorus, silicon, silver, and sodium.

### Results

The materials used are summarized in Table 4; the results of the bond strength tests are summarized in Table 5. There was a nearly statistically significant reduction in bond strength when all glass-treated samples that were bonded with One Step were compared with the negative controls (F=2.57, p=0.051). There was a significant difference among samples bonded with All Bond 3 (F=2.79, p=0.037); where the bond strength was increased for the samples that were glass-treated. When separated by glass type, additional differences became evident: For One Step, the bond strength for 45S5-treated samples was significantly reduced compared to negative controls (F=4.55, p=0.020); this was not the case for samples treated with F glass (F=0.63, p=0.542). Samples that were treated with 45S5 and bonded with All Bond 3 were not significantly different from negative controls (F=0.42, p=0.663), while those treated with F glass and bonded with All Bond 3 exhibited significantly higher bond strength than negative controls (F=3.76, p=0.036). In all cases but one, the standard deviation for glass-treated samples was reduced compared to the negative controls.

The appearance of the samples to which the glass was added differed from the controls. The bonded surface appeared grainy to varying degrees; the shine that is typical after curing was somewhat reduced; and the F glass-treated teeth that were bonded with All Bond 3 were slightly sticky after curing.

TABLE 4
Materials used

| Material | Name | Lot Numbers |
|---|---|---|
| Etchant | UniEtch | 0500001425 |
| Adhesive | One Step | 543-139-A; 543-138A; 543- |
| | | 142-A; 543-142-B |
| Adhesive (primer) | All Bond 3 Primer B | 0700001681 |
| Adhesive | All Bond 3 | part B 543-139-B; part C |
| | | 543-139-C, 543-142-C; |
| | | part D 543-142-D |
| Composite | Aelite All-Purpose Body | 0600010578 |
| | Composite | |
| Composite | Light Core Blue | lot 0600010807 |

TABLE 5
Bond strengths

| Filler Type | Wt % | N | One Step | N | All Bond 3 |
|---|---|---|---|---|---|
| 45S5 | 1 | 10 | 30.4 ± 3.6 | 10 | 33.9 ± 5.9 |
| | 0.5 | 10 | 33.4 ± 3.2 | 10 | 36.2 ± 5.1 |
| F glass | 1 | 10 | 34.6 ± 5.2 | 10 | 34.9 ± 5.4 |
| | 0.5 | 10 | 35.0 ± 4.4 | 10 | 42.2 ± 8.3 |
| None | N/A | 10 | 37.3 ± 7.6 | 10 | 35.4 ± 6.0 |

SEM/EDX analysis revealed formation of an apparently normal hybrid layer for all glass-treated samples. Leakage was evident in negative controls, as shown by areas of high silver concentration. Leakage appeared to be reduced in all samples to which F glass had been added, and did not appear dependent upon filler load or adhesive type; however, samples that had been treated with 45S5 appeared to require a filler load of at least 1 wt % to demonstrate reduced leakage.

The above showed that addition of F glass to an ethanol-based resin bonding system significantly increased bond strength and reduced leakage when compared to negative controls.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method for preparing a tooth for bonding to a dental resin composite, comprising:
applying an etching composition comprising an etchant to a tooth to produce an etched dentin surface;
applying a priming composition comprising a primer to the etched dentin surface;
applying an adhesive composition comprising a resin-based adhesive to the etched and primed dentin surface;
wherein at least one of the adhesive composition or the priming composition comprises a bioactive glass substantially lacking silanol groups and a non-aqueous solvent, and wherein the priming composition and the adhesive composition are optionally combined;
wherein an adhesive layer and a hybrid layer are formed, said hybrid layer comprising said dentin and said resin-based adhesive.

2. The method of claim 1, wherein said primer of the priming composition is a self-etching primer, and the etching composition is optionally not applied in a separate step.

3. The method of claim 1, wherein said resin-based adhesive is a self-etching adhesive, and the etching composition and the priming composition are optionally not applied in separate steps.

4. The method of claim 1, wherein the adhesive composition comprises the primer, and the priming composition is optionally not applied in a separate step.

5. The method of claim 1, wherein said bioactive glass is present at about 0.5% to 10% by weight percentage of the priming composition or the adhesive composition.

6. The method of claim 1, wherein said bioactive glass has the following approximate composition by weight percentage: SiO₂ (44%), Na₂O (23%), CaO (10%), MgO (4.5%), P₂O₅ (6%), and CaF₂ (12.5%).

7. The method of claim 1, wherein said bioactive glass has an average particle size of 1 μm or less.

8. The method of claim 1, wherein said non-aqueous solvent is an alcohol.

9. The method of claim 8, wherein said alcohol is ethanol.

10. The method of claim 1, comprising contacting said adhesive layer with a dental resin composite, wherein said dental resin composite is adhered to said dentin.

11. A method for preparing a tooth for bonding to a dental resin composite, comprising:
applying an etching composition comprising an etchant to a tooth to produce an etched dentin surface;
applying a bioactive glass composition comprising a bioactive glass substantially lacking silanol groups and a non-aqueous solvent;
applying a priming composition comprising a primer to the etched dentin surface; and
applying a adhesive composition comprising a resin-based adhesive to the etched and primed dentin surface;
wherein an adhesive layer and a hybrid layer are formed, said hybrid layer comprising said dentin and said resin-based adhesive.

12. The method of claim 11, wherein said bioactive glass is present at about 0.5% to 40% by weight percentage of the bioactive glass composition.

13. The method of claim 11, wherein said bioactive glass has the following approximate composition by weight percentage: SiO₂ (44%), Na₂O (23%), CaO (10%), MgO (4.5%), P₂O₅ (6%), and CaF₂ (12.5%).

14. The method of claim 11, wherein said bioactive glass has an average particle size of 1 μm or less.

15. The method of claim 11, wherein said non-aqueous solvent is an alcohol.

16. The method of claim 15, wherein said alcohol is ethanol.

17. The method of claim 11, comprising contacting said adhesive layer with a dental resin composite, wherein said dental resin composite is adhered to said dentin.

18. A dental bonding composition comprising:
a bioactive glass substantially lacking silanol groups;
at least one of a resin-based adhesive and a primer; and
a non-aqueous solvent comprising an alcohol.

19. The dental bonding composition of claim 18, wherein the resin-based adhesive is a self-etching adhesive.

20. The dental bonding composition of claim 18, wherein the primer is a self-etching primer.

21. The dental bonding composition of claim 18, wherein the dental bonding composition comprises both the resin-based adhesive and the primer.

22. The dental bonding composition of claim 21, further comprising an etchant.

23. The dental bonding composition of claim 18, wherein said bioactive glass has the following approximate composition by weight percentage: SiO₂ (44%), Na₂O (23%), CaO (10%), MgO (4.5%), P₂O₅ (6%), and CaF₂ (12.5%).

24. The dental bonding composition of claim 18, wherein said bioactive glass has an average particle size of 1 μm or less.

25. The dental bonding composition of claim 18, wherein said adhesive composition comprises about 0.5% to 10% by weight of said bioactive glass.

26. The dental bonding composition of claim 18, wherein said alcohol is ethanol.

27. A kit comprising:
a dental bonding composition comprising
a bioactive glass substantially lacking silanol groups;
at least one of a resin-based adhesive and a primer; and
a non-aqueous solvent comprising an alcohol;
optionally, an etching composition; and
optionally, a resin-based composite suitable for use with the dental bonding composition.

28. The kit according to claim 27, wherein the dental bonding composition is provided as separate components, wherein
a first component comprises the bioactive glass in the non-aqueous solvent, wherein the first component is provided in a first container; and
a second component comprises at least one of the resin-based adhesive and the primer, wherein the second component is provided in a second container.

29. The kit according to claim 27, wherein the dental bonding composition is provided as separate components, wherein
a first component comprises the bioactive glass, the primer, the resin-based adhesive, and the non-aqueous solvent, wherein the first component is provided in a first container.

30. The kit according to claim 27, wherein the dental bonding composition is provided as separate components, wherein
a first component comprises the bioactive glass, the primer, and the non-aqueous solvent, wherein the first component is provided in a first container; and
a second component comprises the resin-based adhesive.

31. A method for making an adhesive composition for bonding a dental resin composite to dentin, comprising:
mixing in a non-aqueous solvent a bioactive glass substantially lacking silanol groups and at least one of a resin-based adhesive and a primer.

32. The method of claim 31, wherein said non-aqueous solvent is an alcohol.
